(19) United States
(12) Patent Application Publication
(10) Pub. No.: US 2026/0206723 A1
(43) Pub. Date: Jul. 23, 2026

(54) CANINE URINE COLLECTION DEVICE

(71) Applicant: Shirley Hobson, Madison, TN (US)

(72) Inventor: Shirley Hobson, Madison, TN (US)

(21) Appl. No.: 19/033,980

(22) Filed: Jan. 22, 2025

(51) Int. Cl. A01K 23/00; A61B 10/00

(52) U.S. Cl. CPC A01K 23/00; A61B 10/007; A61B 2503/40

(57) ABSTRACT

A canine urine collection device includes a first strap that is worn over a torso of a canine. A second strap is coupled to the first strap which extends toward a rear end of the canine. A third strap is coupled to the second strap which extends downwardly over the rear end of the canine at a point located next to a tail of the canine. A fourth strap is coupled to the second strap which extends downwardly over the rear end of the canine at a point located next to the tail of the canine. A collection bag is releasably matable to the first strap, the third strap and the fourth strap such that the collection bag is configured to be positioned beneath the canine to collect urine from the canine when the canine urinates. The collection bag is comprised of a fluid impermeable material to contain the urine.

### (b) CROSS-REFERENCE TO RELATED APPLICATIONS

Not Applicable

### (c) STATEMENT REGARDING FEDERALLY SPONSORED RESEARCH OR DEVELOPMENT

Not Applicable

### (d) THE NAMES OF THE PARTIES TO A JOINT RESEARCH AGREEMENT

Not Applicable

### (e) INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### (f) STATEMENT REGARDING PRIOR DISCLOSURES BY THE INVENTOR OR JOINT INVENTOR

Not Applicable

### (g) BACKGROUND OF THE INVENTION

### (1) Field of the Invention

The disclosure relates to urine collection devices and more particularly pertains to a new urine collection device for collecting urine from a canine. The device includes a first strap and a second strap perpendicular to the first strap and a third strap and a fourth strap which each angle away from the second strap in opposite directions from each other. The first strap is worn over a torso of the canine such that each of the third strap and the fourth strap extend downwardly along a rear end of the canine. The device includes a collection bag that is removably attachable to the first strap and the third strap and the fourth strap such that the collection bag surrounds genitalia of the canine for collecting urine from the canine.

### (2) Description of Related Art Including Information Disclosed Under 37 CFR 1.97 and 1.98

The prior art relates to urine collection devices including: a urine specimen collection device that includes a strap that is wrapped around a torso of a canine and a pouch attached to the strap for collecting urine from the canine; a urine collection device that includes a panel which as a urine opening and a plurality of straps attached to the panel which are wrapped around a body of a canine to position the panel beneath the canine and a urine cup that is removably attachable to the urine opening for collecting a urine sample from the canine; a variety of waste collection devices that each at least includes a harness that can be worn on a body of a canine that includes a plurality of straps and couplers to attach the straps together to form closed loops and a collection cup that is located beneath the canine for collecting waste from the canine; a variety of urine collection devices that each at least includes a strap that is matable to itself to form a closed loop around a body of a canine and a cup attached to the strap to collect urine from the canine. In no instance does the prior art disclose a canine urine collection device that includes a first strap that is worn over a torso of a canine and a second strap that extends along a back of the canine and a third strap and a fourth strap that extend downwardly along a rear end of the canine and a collection bag that is removably attachable to the first strap and the third strap and the fourth strap for collecting a urine sample from the canine.

### (h) BRIEF SUMMARY OF THE INVENTION

An embodiment of the disclosure meets the needs presented above by generally comprising a first strap that is worn over a torso of a canine. A second strap is coupled to the first strap which extends toward a rear end of the canine. A third strap is coupled to the second strap which extends downwardly over the rear end of the canine at a point located next to a tail of the canine. A fourth strap is coupled to the second strap which extends downwardly over the rear end of the canine at a point located next to the tail of the canine. A collection bag is releasably matable to the first strap, the third strap and the fourth strap such that the collection bag is configured to be positioned beneath the canine to collect urine from the canine when the canine urinates. The collection bag is comprised of a fluid impermeable material to contain the urine.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### (i) BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of a canine urine collection device according to an embodiment of the disclosure.

FIG. 2 is a top view of an embodiment of the disclosure.

FIG. 3 is a bottom view of a first strap and a second strap and a third strap and a fourth strap of an embodiment of the disclosure.

FIG. 4 is a top in-use view of an embodiment of the disclosure.

FIG. 5 is a perspective in-use view of an embodiment of the disclosure.

### (j) DETAILED DESCRIPTION OF THE INVENTION

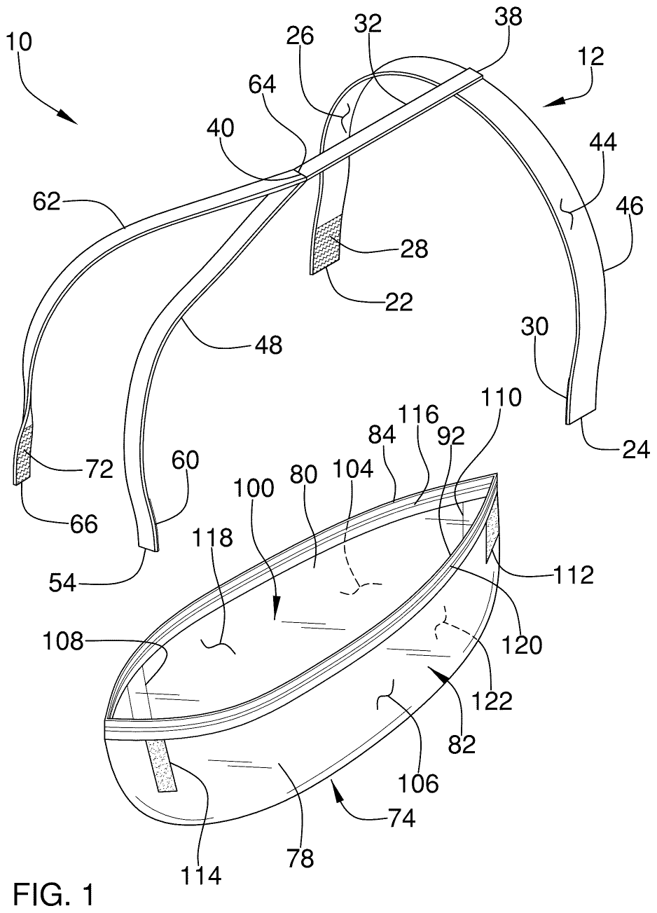

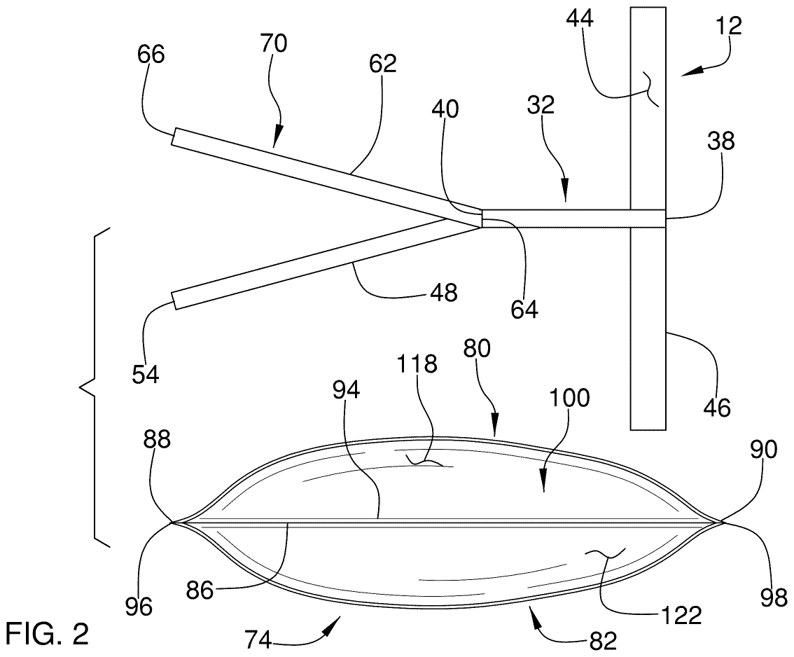

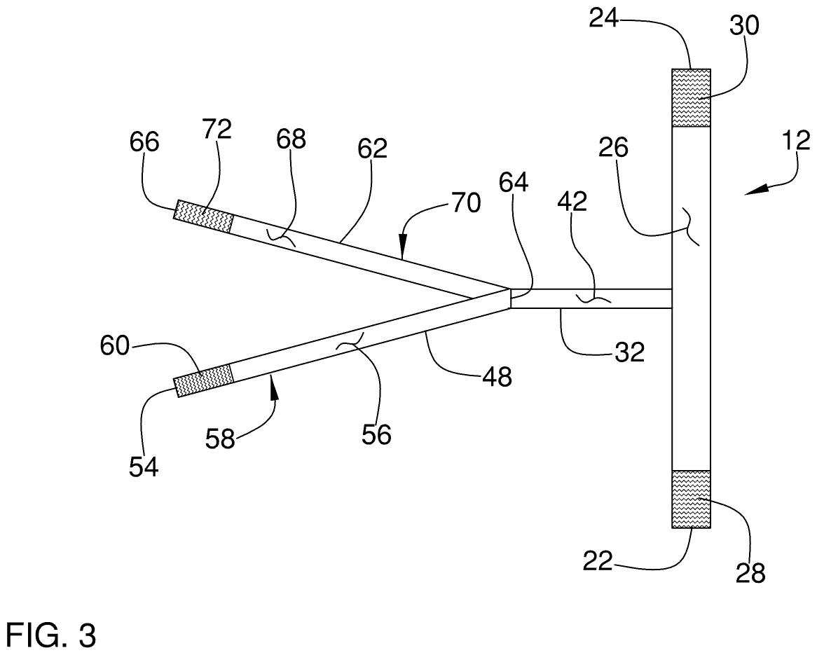

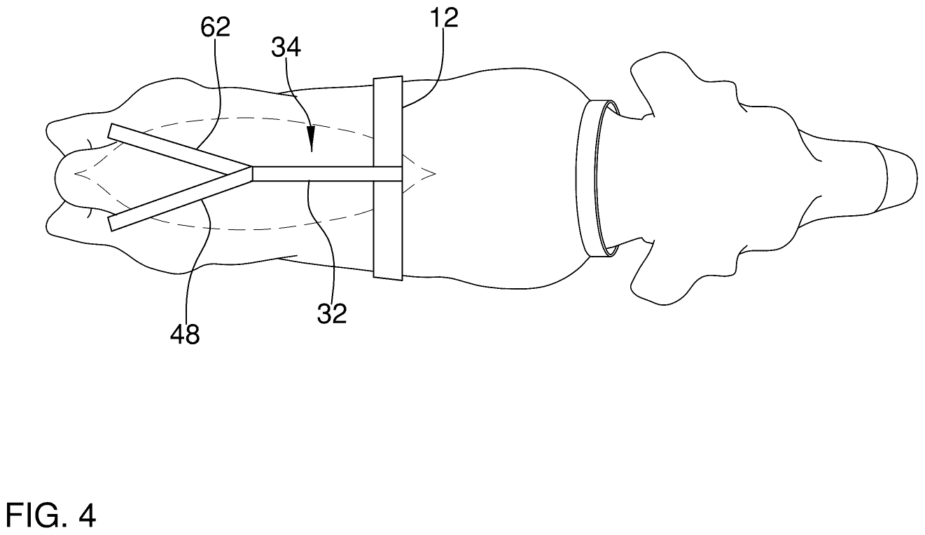

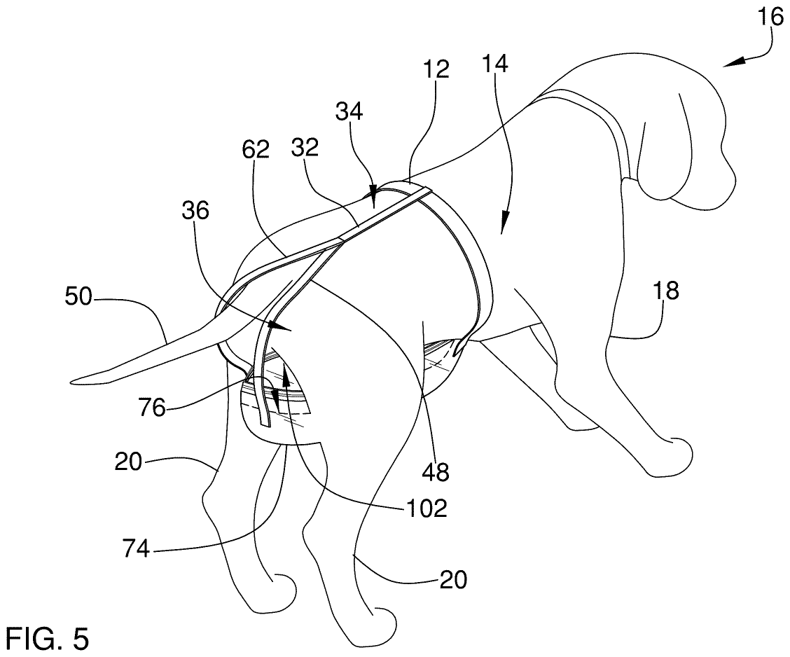

With reference now to the drawings, and in particular to FIGS. 1 through 5 thereof, a new urine collection device embodying the principles and concepts of an embodiment of the disclosure and generally designated by the reference numeral 10 will be described.

As best illustrated in FIGS. 1 through 5, the canine urine collection device 10 generally comprises a first strap 12 which can be worn over a torso 14 of a canine 16 at a point located between front legs 18 of the canine 16 and back legs 20 of the canine 16. Additionally, the canine 16 may be a domesticated dog of any breed. The first strap 12 has a first end 22 and a second end 24 and a lower surface 26 extending between the first end 22 and the second end 24. A first coupler 28 is coupled to the lower surface 26 of the first strap 12 and the first coupler 28 is located adjacent to the first end 22 of the first strap 12. Furthermore, the first coupler 28 may comprise a hook portion of a hook and loop coupler. A second coupler 30 is coupled to the lower surface 26 of the first strap 12 and the second coupler 30 is located adjacent to the second end 24 of the first strap 12. Furthermore, the second coupler 30 may comprise a hook portion of a hook and loop coupler.

A second strap 32 is provided and the second strap 32 is coupled to the first strap 12. The second strap 32 is perpendicularly oriented with the first strap 12 such that the second strap 32 extends along a back 34 of the canine 16 when the first strap 12 is worn over the torso 14 of the canine 16 having the second strap 32 extending toward a rear end 36 of the canine 16. The second strap 32 has a primary end 38 and a secondary end 40 and a bottom surface 42 extending between the primary end 38 and the secondary end 40. The first strap 12 has a top surface 44 extending between the first end 22 and the second end 24 of the first strap 12; the first strap 12 has a leading edge 46 extending between the first end 22 and the second end 24. The bottom surface 42 of the second strap 32 is coupled to the top surface 44 of the first strap 12 such that the primary end 38 is aligned with the leading edge 46 of the first strap 12 such that the lower surface 26 of the first strap 12 can lie on the torso 14 of the canine 16. Additionally, the second strap 32 is centrally located between the first end 22 and the second end 24 of the first strap 12 such that the second strap 32 is aligned with a spine of the canine 16 when the first strap 12 is worn over the torso 14 of the canine 16.

A third strap 48 is coupled to the second strap 32 such that the third strap 48 is distally located with respect to the first strap 12. Furthermore, the third strap 48 lies on the back 34 of the canine 16 when the first strap 12 is worn around the torso 14 of the canine 16. The third strap 48 is oriented to angle away from the second strap 32 such that the third strap 48 extends downwardly over the rear end 36 of the canine 16 at a point located next to a tail 50 of the canine 16. The third strap 48 has a coupled end 52 and a free end 54 and a lowermost surface 56 extending between the coupled end 52 and the free end 54; the third strap 48 has a lateral edge 58 extending between the coupled end 52 and the free end 54. The free end 54 is oriented perpendicular to the lateral edge 58 and the coupled end 52 is oriented at an obtuse angle with the lateral edge 58. Furthermore, the coupled end 52 is coupled to the secondary end 40 of the second strap 32 such that the third strap 48 angles away from the secondary end 40 of the second strap 32 in a first direction. A third coupler 60 is coupled to the lowermost surface 56 of the third strap 48 and the third coupler 60 is located adjacent to the free end 54 of the third strap 48. Additionally, the third coupler 60 may comprise a hook portion of a hook and loop coupler.

A fourth strap 62 is coupled to the second strap 32 such that the fourth strap 62 is distally located with respect to the first strap 12. Furthermore, the fourth strap 62 lies on the back of the canine 16 when the first strap 12 is worn around the torso 14 of the canine 16. The fourth strap 62 is oriented to angle away from the second strap 32 such that the fourth strap 62 extends downwardly over the rear end 36 of the canine 16 at a point located next to the tail 50 of the canine 16. The fourth strap 62 has a coupled end 64 of the fourth strap 62 and a free end 66 of the fourth strap 62 and a lowermost surface 68 of the fourth strap 62 extending between the coupled end 64 of the fourth strap 62 and the free end 66 of the fourth strap 62. Additionally, the fourth strap 62 has a lateral edge 70 of the fourth strap 62 extending between the coupled end 64 of the fourth strap 62 and the free end 66 of the fourth strap 62.

The free end 66 of the fourth strap 62 is oriented perpendicular to the lateral edge 70 of the fourth strap 62. The coupled end 64 of the fourth strap 62 is oriented at an obtuse angle with the lateral edge 70 of the fourth strap 62. Furthermore, the coupled end 64 of the fourth strap 62 is coupled to the secondary end 40 of the second strap 32 such that the fourth strap 62 angles away from the secondary end 40 of the second strap 32 in a second direction such that the third strap 48 and the fourth strap 62 forms a V with the second strap 32. Consequently, each of the third strap 48 and the fourth strap 62 can be positioned on opposite sides of the tail 50 of the canine 16 when the first strap 12 is worn over the torso 14 of the canine 16. In addition, the coupled end 64 of the fourth strap 62 lies on top of the coupled end 52 of the third strap 48. A fourth coupler 72 is coupled to the lowermost surface 68 of the fourth strap 62 and the fourth coupler 72 is located adjacent to the free end 66 of the fourth strap 62. Furthermore, the fourth strap 62 may comprise a hook portion of a hook and loop coupler.

A collection bag 74 is releasably matable to the first strap 12 and the third strap 48 and the fourth strap 62 such that the collection bag 74 is positioned beneath the canine 16 when the first strap 12 is worn over the torso 14 of the canine 16. Consequently, the collection bag 74 can collect urine 76 from the canine 16 when the canine 16 urinates. The collection bag 74 is comprised of a fluid impermeable material, including but not being limited to plastic or silicone or rubber, such that the collection bag 74 can contain the urine 76. The collection bag 74 has an outer wall 78 which has a first lateral side 80 and a second lateral side 82. The first lateral side 80 has a top edge 84 and a bottom edge 86 and a back edge 88 and a front edge 90; the second lateral side 82 has an upper edge 92 and a lower edge 94 and a rear edge 96 and a forward edge 98. The back edge 88 of the first lateral side 80 is coupled to the rear edge 96 of the second lateral side 82 and the front edge 90 of the first lateral side 80 is coupled to the forward edge 98 of the second lateral side 82. Additionally, the bottom edge 86 of the first lateral side 80 is coupled to the lower edge 94 of the second lateral side 82.

The top edge 84 of the first lateral side 80 is spaced from the upper edge 92 of the second lateral side 82 to define an opening 100 into the collection bag 74. Furthermore, the opening 100 extends around genitalia 102 of the canine 16 when the collection bag 74 is releasably mated to the first strap 12 and the third strap 48 and the fourth strap 62. Consequently, the collection bag 74 can collect the urine 76 from the canine 16 without spilling the urine 76 when the canine 16 urinates. The collection bag is elongated between the back edge 88 and front edge 90 of the first lateral side 80 and the rear edge 86 and the forward edge 98 of the second lateral side 82.

The first lateral side 80 has an outwardly facing surface 104 and the second lateral side 82 has an outwardly facing surface 106 of the second lateral side 82. A fifth coupler 108 is coupled to the outwardly facing surface 104 of the first lateral side 80 of the outer wall 78 of the bag. The fifth coupler 108 is spaced from an intersection between the back edge 88 of the first lateral side 80 and the rear edge 96 of the second lateral side 82 of the outer wall 78 of the collection bag 74. Additionally, the fifth coupler 108 is oriented to extend along a line extending between the top edge 84 of the first lateral side 80 and an intersection between the bottom edge 86 of the first lateral side 80 and the lower edge 94 of the second lateral side 82. The fourth coupler 72 is releasably matable to the fifth coupler 108 and the fifth coupler 108 may comprise a loop portion of a hook and loop coupler.

A sixth coupler 110 is coupled to the outwardly facing surface 104 of the first lateral side 80 of the outer wall 78 of the bag. The sixth coupler 110 is spaced from an intersection between the back edge 88 of the first lateral side 80 and the rear edge 96 of the second lateral side 82 of the outer wall 78 of the collection bag 74. Furthermore, the sixth coupler 110 is oriented to extend along a line extending between the top edge 84 of the first lateral side 80 and an intersection between the bottom edge 86 of the first lateral side 80 and the lower edge 94 of the second lateral side 82. The first coupler 28 is releasably matable to the sixth coupler 110 and the sixth coupler 110 may comprise a loop portion of a hook and loop coupler.

A seventh coupler 112 is coupled to the outwardly facing surface 106 of the second lateral side 82 of the outer wall 78 of the bag. The seventh coupler 112 is spaced from an intersection between the front edge 90 of the first lateral side 80 of the outer wall 78 of the collection bag 74 and the forward edge 98 of the second lateral side 82. Additionally, the seventh coupler 112 is oriented to extend along a line extending between the upper edge 92 of the second lateral side 82 and an intersection between the bottom edge 86 of the first lateral side 80 and the lower edge 94 of the second lateral side 82. The seventh coupler 112 is aligned with the sixth coupler 110 and the second coupler 30 is releasably matable to the seventh coupler 112. Additionally, the seventh coupler 112 may comprise a loop portion of a hook and loop coupler.

An eighth coupler 114 is coupled to the outwardly facing surface 106 of the second lateral side 82 of the outer wall 78 of the bag. The eighth coupler 114 is spaced from an intersection between the back edge 88 of the first lateral side 80 of the outer wall 78 of the collection bag 74 and the rear edge 96 of the second lateral side 82. Additionally, the eighth coupler 114 is oriented to extend along a line extending between the upper edge 92 of the second lateral side 82 and an intersection between the bottom edge 86 of the first lateral side 80 and the lower edge 94 of the second lateral side 82. The eighth coupler 114 is aligned with the fifth coupler 108 and the second coupler 30 is releasably matable to the eighth coupler 114. Furthermore, the eighth coupler 114 may comprise a loop portion of a hook and loop coupler.

A first closure 116 is provided and the first closure 116 is coupled to the collection bag 74. The first lateral side 80 of the outer wall 78 of the collection bag 74 has an inwardly facing surface 118 and the first closure 116 is located on the inwardly facing surface 118. The first closure 116 extends between the back edge 88 of the first lateral side 80 and the front edge 90 of the first lateral side 80. Furthermore, the first closure 116 is aligned with the top edge 84 of the first lateral side 80.

A second closure 120 is provided and the second closure 120 is coupled to the collection bag 74. The second closure 120 is releasably matable to the first closure 116 thereby closing the collection bag 74 to retain the urine 76 in the collection bag 74. The second lateral side 82 of the outer wall 78 of the collection bag 74 has an inwardly facing surface 122 of the second lateral side 82 and the second closure 120 is located on the inwardly facing surface 122 of the second lateral side 82. Additionally, the second closure 120 extends between the rear edge 96 of the second lateral side 82 and the forward edge 98 of the second lateral side 82 and the second closure 120 is aligned with the upper edge 92 of the second lateral side 82. The first closure 116 forms a fluid impermeable seal with the second closure 120 when the first closure 116 is releasably mated to the second closure 120 to inhibit the urine 76 from leaking out of the collection bag 74. Each of the first closure 116 and the second closure 120 may comprise complementary portions of a zip type closure or other type of closure that is capable of forming a fluid impermeable seal.

In use, the first strap 12 is positioned on the canine 16 such that the first strap 12 extends downwardly around the torso 14 of the canine 16 and the second strap 32 extends toward the rear end 36 of the canine 16 when a urine sample is required to be collected from the canine 16. Furthermore, each of the third strap 48 and the fourth strap 62 extend downwardly along the rear end 36 of the canine 16 on opposite sides of the tail 50 of the canine 16. The collection bag 74 is removably coupled to each of the first strap 12 and the third strap 48 and the second strap 32 such that the collection bag 74 surrounds the genitalia 102 of the canine 16. The collection bag 74 collects urine 76 from the canine 16 when the canine 16 urinates and the collection bag 74 is subsequently removed from each of the first strap 12 and the third strap 48 and the fourth strap 62. The first closure 116 is mated to the second closure 120 to close the collection bag 74 and retain the urine 76 in the collection bag 74. Subsequently, the collection bag 74 is delivered to a veterinarian such that the veterinarian can perform testing on the urine 76 to diagnose health problems of the canine 16.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A canine urine collection device for collecting urine from a canine, said device comprising:
a first strap being configured to be worn over a torso of a canine at a point located between front legs of the canine and back legs of the canine;
a second strap being coupled to said first strap, said second strap being perpendicularly oriented with said first strap wherein said second strap is configured to extend along a back of the canine when said first strap is worn over the torso of the canine having said second strap extending toward a rear end of the canine;
a third strap being coupled to said second strap such that said third strap is distally located with respect to said first strap wherein said third strap is configured to lie on the back of the canine when said first strap is worn around the torso of the canine, said third strap being oriented to angle away from said second strap wherein said third strap is configured to extend downwardly over the rear end of the canine at a point located next to a tail of the canine;
a fourth strap being coupled to said second strap such that said fourth strap is distally located with respect to said first strap wherein said fourth strap is configured to lie on the back of the canine when said first strap is worn around the torso of the canine, said fourth strap being oriented to angle away from said second strap wherein said fourth strap is configured to extend downwardly over the rear end of the canine at a point located next to the tail of the canine; and
a collection bag being releasably matable to said first strap and said third strap and said fourth strap wherein said collection bag is configured to be positioned beneath the canine when said first strap is worn over the torso of the canine such that said collection bag can collect urine from the canine when the canine urinates, said collection bag being comprised of a fluid impermeable material wherein said collection bag is configured to contain the urine.

2. The device according to claim 1, wherein:
said first strap having a first end and a second end and a lower surface extending between said first end and said second end;
said device includes a first coupler being coupled to said lower surface of said first strap;
said first coupler is located adjacent to said first end of said first strap;
said device includes a second coupler being coupled to said lower surface of said first strap; and
said second coupler is located adjacent to said second end of said first strap.

3. The device according to claim 1, wherein:
said second strap has a primary end and a secondary end and a bottom surface extending between said primary end and said secondary end;
said first strap has a top surface extending between a first end and a second end of said first strap;
said first strap has a lower surface extending between said first end and said second end;
said first strap has a leading edge extending between said first end and said second end;
said bottom surface of said second strap is coupled to said top surface of said first strap such that said primary end is aligned with said leading edge of said first strap wherein said lower surface of said first strap is configured to lie on the torso of the canine; and
said second strap is centrally located between said first end and said second end of said first strap wherein said second strap is configured to be aligned with a spine of the canine when said first strap is worn over the torso of the canine.

4. The device according to claim 3, wherein:
said third strap has a coupled end and a free end and a lowermost surface extending between said coupled end and said free end;
said third strap has a lateral edge extending between said coupled end and said free end;
said free end is oriented perpendicular to said lateral edge;
said coupled end is oriented at an obtuse angle with said lateral edge;
said coupled end is coupled to said secondary end of said second strap;
said device includes a third coupler being coupled to said lowermost surface of said third strap; and
said third coupler is located adjacent to said free end of said third strap.

5. The device according to claim 3, wherein:
said fourth strap has a coupled end of said fourth strap and a free end of said fourth strap and a lowermost surface of said fourth strap extending between said coupled end of said fourth strap and said free end of said fourth strap;
said fourth strap has a lateral edge of said fourth strap extending between said coupled end of said fourth strap and said free end of said fourth strap;
said free end of said fourth strap is oriented perpendicular to said lateral edge of said fourth strap;
said coupled end of said fourth strap is oriented at an obtuse angle with said lateral edge of said fourth strap;
said coupled end of said fourth strap is coupled to said secondary end of said second strap;
said device includes a fourth coupler being coupled to said lowermost surface of said fourth strap; and
said fourth coupler is located adjacent to said free end of said fourth strap.

6. The device according to claim 1, wherein:
said second strap has a primary end and a secondary end;
said third strap angles away from said secondary end of said second strap in a first direction; and
said fourth strap angles away from said secondary end of said second strap in a second direction such that said third strap and said fourth strap forms a V with said second strap wherein each of said third strap and said fourth strap are configured to be positioned on opposite sides of the tail of the canine when said first strap is worn over the torso of the canine.

7. The device according to claim 1, wherein:
said collection bag has an outer wall which has a first lateral side and a second lateral side;
said first lateral side has a top edge and a bottom edge and a back edge and a front edge;
said second lateral side has an upper edge and a lower edge and a rear edge and a forward edge;
said back edge of said first lateral side is coupled to said rear edge of said second lateral side;
said front edge of said first lateral side is coupled to said forward edge of said second lateral side;
said bottom edge of said first lateral side is coupled to said lower edge of said second lateral side; and
said top edge of said first lateral side is spaced from said upper edge of said second lateral side to define an opening into said collection bag wherein said opening is configured to extend around genitalia of the canine when said collection bag is releasably mated to said first strap and said third strap and said fourth strap such that said collection bag can collect the urine from the canine without spilling the urine when the canine urinates.

8. The device according to claim 7, wherein:
said first lateral side has an outwardly facing surface;
said second lateral side has an outwardly facing surface of said second lateral side;
said device includes a first coupler being coupled to said first strap;
said device includes a second coupler being coupled to said first strap;
said device includes a third coupler being coupled to said third strap;
said device includes a fourth coupler being coupled to said fourth strap;
said device includes a fifth coupler being coupled to said outwardly facing surface of said first lateral side of said outer wall of said bag;
said device includes a sixth coupler being coupled to said outwardly facing surface of said first lateral side of said outer wall of said bag;
said device includes a seventh coupler being coupled to said outwardly facing surface of said second lateral side of said outer wall of said bag; and
said device includes an eighth coupler being coupled to said outwardly facing surface of said second lateral side of said outer wall of said bag.

9. The device according to claim 8, wherein:
said fifth coupler is spaced from an intersection between said back edge of said first lateral side and said rear edge of said second lateral side of said outer wall of said collection bag;
said fifth coupler is oriented to extend along a line extending between said top edge of said first lateral side and an intersection between said bottom edge of said first lateral side and said lower edge of said second lateral side.

10. The device according to claim 8, wherein said fourth coupler is releasably matable to said fifth coupler.

11. The device according to claim 8, wherein:
said sixth coupler is spaced from an intersection between said back edge of said first lateral side and said rear edge of said second lateral side of said outer wall of said collection bag; and
said sixth coupler is oriented to extend along a line extending between said top edge of said first lateral side and an intersection between said bottom edge of said first lateral side and said lower edge of said second lateral side.

12. The device according to claim 8, wherein said first coupler is releasably matable to said sixth coupler.

13. The device according to claim 8, wherein:
said seventh coupler is spaced from an intersection between said front edge of said first lateral side of said outer wall of said collection bag and said forward edge of said second lateral side;
said seventh coupler is oriented to extend along a line extending between said upper edge of said second lateral side and an intersection between said bottom edge of said first lateral side and said lower edge of said second lateral side;
said seventh coupler is aligned with said sixth coupler; and
said second coupler is releasably matable to said seventh coupler.

14. The device according to claim 8, wherein:
said eighth coupler is spaced from an intersection between said back edge of said first lateral side of said outer wall of said collection bag and said rear edge of said second lateral side;
said eighth coupler is oriented to extend along a line extending between said upper edge of said second lateral side and an intersection between said bottom edge of said first lateral side and said lower edge of said second lateral side;
said eighth coupler is aligned with said fifth coupler; and
said second coupler is releasably matable to said eighth coupler.

15. The device according to claim 8, wherein:
said device includes a first closure being coupled to said collection bag;
said first lateral side of said outer wall of said collection bag has an inwardly facing surface;
said first closure is located on said inwardly facing surface;
said first closure extends between said back edge of said first lateral side and said front edge of said first lateral side; and
said first closure is aligned with said top edge of said first lateral side.

16. The device according to claim 15, wherein:
said device includes a second closure being coupled to said collection bag;
said second closure is releasably matable to said first closure thereby closing said collection bag wherein each of said first closure and said second closure is configured to retain the urine in said collection bag;
said second lateral side of said outer wall of said collection bag has an inwardly facing surface of said second lateral side;
said second closure is located on said inwardly facing surface of said second lateral side;
said second closure extends between said rear edge of said second lateral side and said forward edge of said second lateral side; and
said second closure is aligned with said upper edge of said second lateral side.

17. The device according to claim 16, wherein said first closure forms a fluid impermeable seal with said second closure when said first closure is releasably mated to said second closure wherein each of said first closure and said second closure are configured to inhibit the urine from leaking out of said collection bag.

18. A canine urine collection device for collecting urine from a canine, said device comprising:
a first strap being configured to be worn over a torso of a canine at a point located between front legs of the canine and back legs of the canine, said first strap having a first end and a second end and a lower surface extending between said first end and said second end;
a first coupler being coupled to said lower surface of said first strap, said first coupler being located adjacent to said first end of said first strap;
a second coupler being coupled to said lower surface of said first strap, said second coupler being located adjacent to said second end of said first strap;
a second strap being coupled to said first strap, said second strap being perpendicularly oriented with said first strap wherein said second strap is configured to extend along a back of the canine when said first strap is worn over the torso of the canine having said second strap extending toward a rear end of the canine, said second strap having a primary end and a secondary end and a bottom surface extending between said primary end and said secondary end, said first strap having a top surface extending between said first end and said second end of said first strap, said first strap having a leading edge extending between said first end and said second end, said bottom surface of said second strap being coupled to said top surface of said first strap such that said primary end is aligned with said leading edge of said first strap wherein said lower surface of said first strap is configured to lie on the torso of the canine, said second strap being centrally located between said first end and said second end of said first strap wherein said second strap is configured to be aligned with a spine of the canine when said first strap is worn over the torso of the canine;
a third strap being coupled to said second strap such that said third strap is distally located with respect to said first strap wherein said third strap is configured to lie on the back of the canine when said first strap is worn around the torso of the canine, said third strap being oriented to angle away from said second strap wherein said third strap is configured to extend downwardly over the rear end of the canine at a point located next to a tail of the canine, said third strap having a coupled end and a free end and an lowermost surface extending between said coupled end and said free end, said third strap having a lateral edge extending between said coupled end and said free end, said free end being oriented perpendicular to said lateral edge, said coupled end being oriented at an obtuse angle with said lateral edge, said coupled end being coupled to said secondary end of said second strap such that said third strap angles away from said secondary end of said second strap in a first direction;
a third coupler being coupled to said lowermost surface of said third strap, said third coupler being located adjacent to said free end of said third strap;
a fourth strap being coupled to said second strap such that said fourth strap is distally located with respect to said first strap wherein said fourth strap is configured to lie on the back of the canine when said first strap is worn around the torso of the canine, said fourth strap being oriented to angle away from said second strap wherein said fourth strap is configured to extend downwardly over the rear end of the canine at a point located next to the tail of the canine, said fourth strap having a coupled end of said fourth strap and a free end of said fourth strap and an lowermost surface of said fourth strap extending between said coupled end of said fourth strap and said free end of said fourth strap, said fourth strap having a lateral edge of said fourth strap extending between said coupled end of said fourth strap and said free end of said fourth strap, said free end of said fourth strap being oriented perpendicular to said lateral edge of said fourth strap, said coupled end of said fourth strap being oriented at an obtuse angle with said lateral edge of said fourth strap, said coupled end of said fourth strap being coupled to said secondary end of said second strap such that said fourth strap angles away from said secondary end of said second strap in a second direction such that said third strap and said fourth strap forms a V with said second strap wherein each of said third strap and said fourth strap are configured to be positioned on opposite sides of the tail of the canine when said first strap is worn over the torso of the canine, said coupled end of said fourth strap lying on top of said coupled end of said third strap;
a fourth coupler being coupled to said lowermost surface of said fourth strap, said fourth coupler being located adjacent to said free end of said fourth strap;
a collection bag being releasably matable to said first strap and said third strap and said fourth strap wherein said collection bag is configured to be positioned beneath the canine when said first strap is worn over the torso of the canine such that said collection bag can collect urine from the canine when the canine urinates, said collection bag being comprised of a fluid impermeable material wherein said collection bag is configured to contain the urine, said collection bag having an outer wall which has a first lateral side and a second lateral side, said first lateral side having a top edge and a bottom edge and a back edge and a front edge, said second lateral side having an upper edge and a lower edge and a rear edge and a forward edge, said back edge of said first lateral side being coupled to said rear edge of said second lateral side, said front edge of said first lateral side being coupled to said forward edge of said second lateral side, said bottom edge of said first lateral side being coupled to said lower edge of said second lateral side, said top edge of said first lateral side being spaced from said upper edge of said second lateral side to define an opening into said collection bag wherein said opening is configured to extend around genitalia of the canine when said collection bag is releasably mated to said first strap and said third strap and said fourth strap such that said collection bag can collect the urine from the canine without spilling the urine when the canine urinates, said first lateral side having an outwardly facing surface, said second lateral side having an outwardly facing surface of said second lateral side;
a fifth coupler being coupled to said outwardly facing surface of said first lateral side of said outer wall of said bag, said fifth coupler being spaced from an intersection between said back edge of said first lateral side and said rear edge of said second lateral side of said outer wall of said collection bag, said fifth coupler being oriented to extend along a line extending between said top edge of said first lateral side and an intersection between said bottom edge of said first lateral side and said lower edge of said second lateral side, said fourth coupler being releasably matable to said fifth coupler;
a sixth coupler being coupled to said outwardly facing surface of said first lateral side of said outer wall of said bag, said sixth coupler being spaced from an intersection between said back edge of said first lateral side and said rear edge of said second lateral side of said outer wall of said collection bag, said sixth coupler being oriented to extend along a line extending between said top edge of said first lateral side and an intersection between said bottom edge of said first lateral side and said lower edge of said second lateral side, said first coupler being releasably matable to said sixth coupler;
a seventh coupler being coupled to said outwardly facing surface of said second lateral side of said outer wall of said bag, said seventh coupler being spaced from an intersection between said front edge of said first lateral side of said outer wall of said collection bag and said forward edge of said second lateral side, said seventh coupler being oriented to extend along a line extending between said upper edge of said second lateral side and an intersection between said bottom edge of said first lateral side and said lower edge of said second lateral side, said seventh coupler being aligned with said sixth coupler, said second coupler being releasably matable to said seventh coupler;
a eighth coupler being coupled to said outwardly facing surface of said second lateral side of said outer wall of said bag, said eighth coupler being spaced from an intersection between said back edge of said first lateral side of said outer wall of said collection bag and said rear edge of said second lateral side, said eighth coupler being oriented to extend along a line extending between said upper edge of said second lateral side and an intersection between said bottom edge of said first lateral side and said lower edge of said second lateral side, said eighth coupler being aligned with said fifth coupler, said second coupler being releasably matable to said eighth coupler;
a first closure being coupled to said collection bag, said first lateral side of said outer wall of said collection bag having an inwardly facing surface, said first closure being located on said inwardly facing surface, said first closure extending between said back edge of said first lateral side and said front edge of said first lateral side, said first closure being aligned with said top edge of said first lateral side; and
a second closure being coupled to said collection bag, said second closure being releasably matable to said first closure thereby closing said collection bag wherein each of said first closure and said second closure is configured to retain the urine in said collection bag, said second lateral side of said outer wall of said collection bag having an inwardly facing surface of said second lateral side, said second closure being located on said inwardly facing surface of said second lateral side, said second closure extending between said rear edge of said second lateral side and said forward edge of said second lateral side, said second closure being aligned with said upper edge of said second lateral side, said first closure forming a fluid impermeable seal with said second closure when said first closure is releasably mated to said second closure wherein each of said first closure and said second closure are configured to inhibit the urine from leaking out of said collection bag.
